(19) United States
(12) Patent Application Publication
(10) Pub. No.: US 2018/0293980 A1
(43) Pub. Date: Oct. 11, 2018

(54) VISUALLY IMPAIRED AUGMENTED REALITY

(71) Applicants: Kumar Narasimhan Dwarakanath, Folsom, CA (US); Moorthy Rajesh, Folsom, CA (US); Senaka Cuda Bandara Ratnayake, El Dorado Hills, CA (US)

(72) Inventors: Kumar Narasimhan Dwarakanath, Folsom, CA (US); Moorthy Rajesh, Folsom, CA (US); Senaka Cuda Bandara Ratnayake, El Dorado Hills, CA (US)

(21) Appl. No.: 15/479,916

(22) Filed: Apr. 5, 2017

(51) Int. Cl. G10L 15/22; G10L 15/26; A61F 9/08

(52) U.S. Cl. CPC G10L 15/22; G10L 2015/223; A61F 9/08; G10L 15/265

(57) ABSTRACT

System and techniques for visually impaired augmented reality are described herein. An utterance may be received from a user. The utterance may be classified to produce a filter. The user's environment may be classified based on the filter to produce an environmental event. An audible interpretation of the environmental event may be rendered.

### TECHNICAL FIELD

Embodiments described herein generally relate to augmented reality equipment and more specifically to visually impaired augmented reality.

### BACKGROUND

Augmented reality (AR) and virtual reality (VR) encompass a number of technologies that interface with a user's senses to modify the real world from the user's perspective. AR often involves modifying an aspect of the real world, such as overlaying graphical information (e.g., directions) onto a scene of the real world. Technologies involved in implementing AR include a variety of sensors to sense the real world and a variety of renders, such as graphical displays or speakers, to effectuate the modification of the real world from the user's perspective.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a block diagram of an example of an environment including a system for visually impaired augmented reality, according to an embodiment.

FIG. 2 illustrates an example navigation for a user, according to an embodiment.

FIG. 3 illustrates an example of a technique to facilitate visually impaired augmented reality, according to an embodiment.

FIG. 4 illustrates an example matrix of a system for visually impaired augmented reality, according to an embodiment.

FIG. 5 illustrates an example of a control stack for visually impaired augmented reality, according to an embodiment.

FIG. 6 illustrates an example of a communications flow for visually impaired augmented reality, according to an embodiment.

FIGS. 7-10 illustrate several examples of sensor arrangements in a wearable device to facilitate visually impaired augmented reality.

FIG. 11 illustrates a flow diagram of an example of a method for visually impaired augmented reality, according to an embodiment.

FIG. 12 is a block diagram illustrating an example of a machine upon which one or more embodiments may be implemented.

### DETAILED DESCRIPTION

AR systems are primarily directed to sighted people with a significant rendering device of many AR systems being a display. Other renderer's, such as speakers, or haptic feedback devices, are generally used to augment the visual display of AR systems. This focus on vision may leave out the visually impaired (e.g., blind persons), a segment of the population that may benefit greatly from AR in navigation and avoiding dangerous or uncomfortable environments.

Blind or visually impaired people generally rely on audible sounds to discern their surroundings. However many times there is no audible input available. For example, while walking down a street, there is generally no audible signaling about restaurants or special pricing on menu items in stores akin to the visual information generally conveyed to sighted persons via billboards or other signage. Aside from commercial obstacles, visually impaired people may encounter a number of scenarios in which senses other than vision are relied upon but may fall short, such as a hole in the sidewalk, or even a puddle in their path. Service animals may provide some help, but often cannot perceive many obstacles or tackle other issues (e.g., reading commercial of civic signage).

To address these issues, AR enabled devices are herein described that provide more context and awareness to a visually impaired user of wearable devices. These devices operate to empower visually impaired people with contextual awareness of their surroundings, as well as address user interface issues with a low information density communications medium, such as voice based AR. The described AR device may be wearable (e.g., head mounted) and integrate several sensors, such as a camera array, a microphone array, a positioning system (e.g., the Global Positioning System (GPS) or other satellite based system, cellular position systems, etc.), radio receivers (e.g., radio frequency identification (RFID) devices or other near field communication (NFC) devices), gyrometers (e.g., gyroscopes), accelerometers, thermometers, altimeters, etc., to perceive the natural (e.g., real) world and provide an audible (e.g., voice-based) or haptic output to facilitate navigation or obstacle awareness/avoidance.

An issue that may arise with audible or haptic guidance is the low-information density of these communication mediums. For example, a running dialogue from the system describing every detail of an environment (e.g., what direction to head, that there is a pothole two feet to the right, five people are approaching from the rear, traffic on the street a block away is light, etc.) may be overwhelming and ultimately useless to the user. To address this issue, the described AR system creates filters based on user instruction to determine what sensor inputs to react upon, as well as what output to provide. In an example, the filter is based on a task assigned by the user. The task may be communicated via a voice command. For example, a user walking down a busy street may determine that she is capable of staying on the sidewalk and avoiding people and trees without additional assistance. However, as it has just rained, the user would like to avoid puddles. Accordingly, the user may provide the command “avoid puddles” to the AR system. The AR system may then modify itself to select sensors capable of detecting puddles and provide puddle avoid direction. Not only does this technique reduce sensory clutter to the user (e.g., overstimulation), it also provides power savings, which may be useful for power constrained wearable devices.

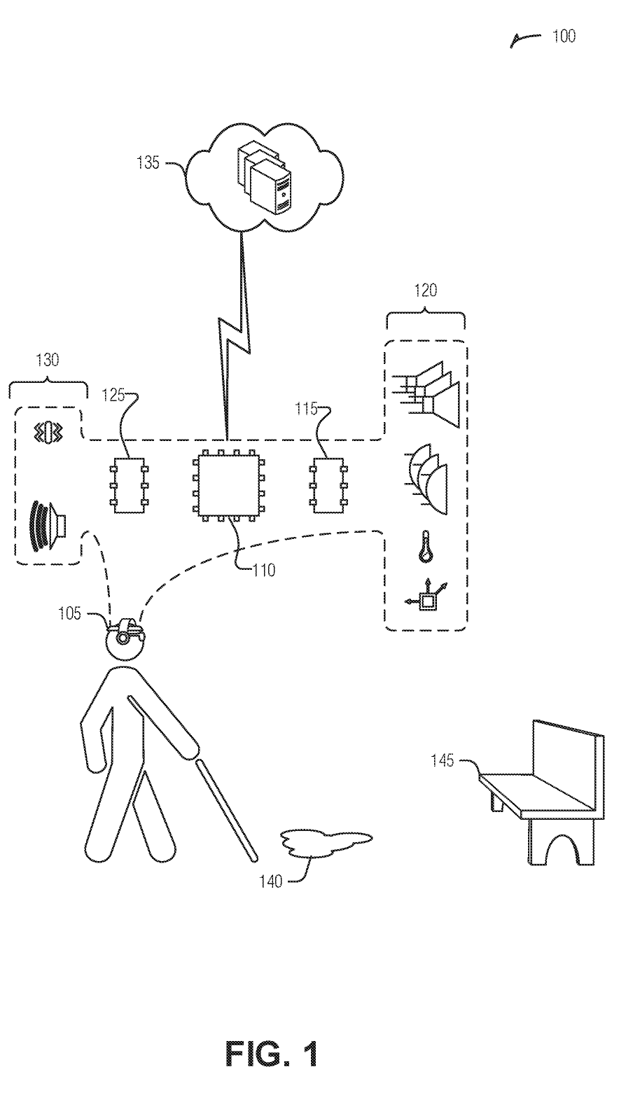

FIG. 1 is a block diagram of an example of an environment 100 including a system 105 for visually impaired AR, according to an embodiment. The environment 100 may also include an optional cloud service 135 communicatively coupled to the system 105 when in operation, an obstacle 140, and a destination 145.

The system 105 includes a controller 110, a sensor interface 115, and an output driver 125. These components 110, 115, 125 of the system 105 are implemented in computer hardware, such as circuitry. The system 105 may be part of (e.g., embedded into) a user wearable or user held device. In an example, the wearable device is head worn. In an example, the head worn wearable is in the form of a circlet, headband, ring, or otherwise encircles the user's head.

The sensor interface 115 provides hardware support to direct or receive information from a variety of sensors 120. In an example, the sensors 120 include one or more cameras. In an example, the sensor 120 include one or more microphones, in an example, the sensors 120 include a thermometer. In an example, the sensors 120 include an accelerometer.

The sensor interface 115 is arranged to receive an utterance from the user. In an example, the utterance is a part of speech, such as a word, or phrase, or sentence. The sensor interface 115 provides (e.g., sends are responds to a request for) the utterance to the controller 110.

The controller 110 is arranged to classify the utterance to produce a filter. Utterance classification may include performing a speech to text transformation on the utterance. The controller 110 may then select a filter based on the text. In an example, the utterance is directly classified to a filter. Such may be accomplished via a trained artificial neural network (ANN). In an example, a portion of the utterance may designate a parameterized filter while other portions of the utterance provides the parameter. For example, the utterance “avoid white dogs” may be parsed to identify an avoidance filter with the target (e.g., to avoid) parameterized as ‘white’ and ‘dog(s)’. In an example, a parameter is ignored if not supported by the classifier (discussed below). For example, the classifier may be trained to recognize dogs but not distinguish the color of a dog. Thus, the parameters ‘white’ would be ignored while the parameter ‘dog(s)’ would not.

In an example, the filter is a command to navigate to a destination. For example, the user may wish to navigate to the bench 145. In this example, the filter may be ‘navigate to the nearest bench’. Other destination designations may include a business name or type (e.g., coffee shop), a street address or intersection, or other designation (e.g., ‘five miles east of County Road 35 on Pleasant Path Lane’).

The controller 110 is arranged to classify the environment 100 of the user based on the filter to produce an environmental event. The controller 110 may implement a classifier or the controller 110 may invoke an external classifier. In an example, the external classifier resides in the system 105. In an example, the external classifier resides in the cloud 135. The classifier accepts input (e.g., from the sensor interface 115 generated or derived from the sensors 120) and produces an output. Environmental classification is often multidisciplinary, employing sensor 120 processing techniques (e.g., color, hue, saturation, etc. adjustments on images, noise filtering on audio, etc.) as well as computer-based decision making techniques, such as ANNs, expert systems, etc. Here, the classifier is arranged to produce an actionable output. Example classifier outputs may include identifying an object (e.g., animate or inanimate), identifying a path to a destination, identifying a condition (e.g., a busy road), among other things. Here, the output of the classifier as modified by the filter is an environmental event. In an example, the environmental event is a waypoint to the destination.

In an example, the classifier is selected from multiple classifiers to classify the environment based on the parameter. Thus, one classifier may be used for navigation while another classifier is used to avoid obstacles 140. This flexibility not only efficiently uses possibly limited computing or power resources on the system 105, but also increases modularity in what types of AR experiences the system 105 provides to the user.

The controller 110 is arranged to render (e.g., via the output driver 125) an interpretation of the environmental event using an output device 130 (e.g., via a haptic feedback device, speaker, or the like). In an example, the interpretation is audible (e.g., an audible interpretation). In an example, the audible interpretation includes a set of words intelligible by the user. This example contrasts with audible events, such as beeps, animal noises (e.g., roar, scream, purr, etc.), or other non-language signals that may be provided to the user. Instead, this example is discernable by the user, such as ‘puddle in five feet straight ahead’ or ‘stop!’

In an example, classifying the environment to produce an environmental event includes limiting a frequency of environmental events produced. This may be important to avoid confusing the user. That is, a certain information density is assumed to be the maximum for audible signaling to still be intelligible to the user. In an example, the frequency of environmental events produced is based on a cardinality of the set of words corresponding to potential events. Thus, shorter phrases may be repeated more often. In an example, the frequency of environmental events produced is based on a time-to-render of the set of words corresponding to potential events. In an example, the frequency of environmental events produced is beyond a threshold. Here, the audible interpretation may be generalized to a description of multiple environmental events. For example, if the filter is based on ‘avoid puddles’, the audible signal may warn of an upcoming puddle so that the user may avoid the puddle. If, however, is has just rained and the street is now very puddled, instead of denoting each puddle, the audible signal may convert to ‘the road is covered in puddles’. Here, the puddle classification continues as before, yet the number of puddles causes individual puddle information to exceed the information density of the audible signaling mechanism, thus the conversion of the audible signal.

In an example, receiving the utterance from the user, classifying the utterance to produce the filter, classifying the environment of the user to produce the environmental event, and rendering the audible interpretation of the environmental event are performed on the set of devices carried by the user. This is the example illustrated in FIG. 1 if the cloud 135 is not used. In an example, the set of devices has a cardinality of one (there is only one device performing these tasks). In an example, the set of devices include at least one of a microphone, a camera, a depth sensor, a distance sensor, a positioning system, a motion sensor, or a context sensor. In an example, the context sensor is at least one of a mapping system, or a short-range radio frequency interrogator.

The following use case uses the system 105 may provide additional context to the elements discussed above. The user decides to walk to a nearby restaurant. The user provides a destination to the system 105 via voice commands and requests navigation to the destination. The system 105 detects the voice command and starts processing contextual inputs from the sensors 120 (e.g., camera, microphone, GPS location, maps, motion sensor (For example it calculates the number of steps the person has to take), etc.) The system 105 then directs the user through audio ques. If requested by the user, the system 105 may also identify and signal obstacles 140 for the user to avoid. In an example, the system 105 may identifying acquaintances that happen to walk by, available stores being passed, etc. In an example, when the user reaches the restaurant, the system may read signage, such as a posted menu, or a menu handed to the user, to further augment the user's AR experience.

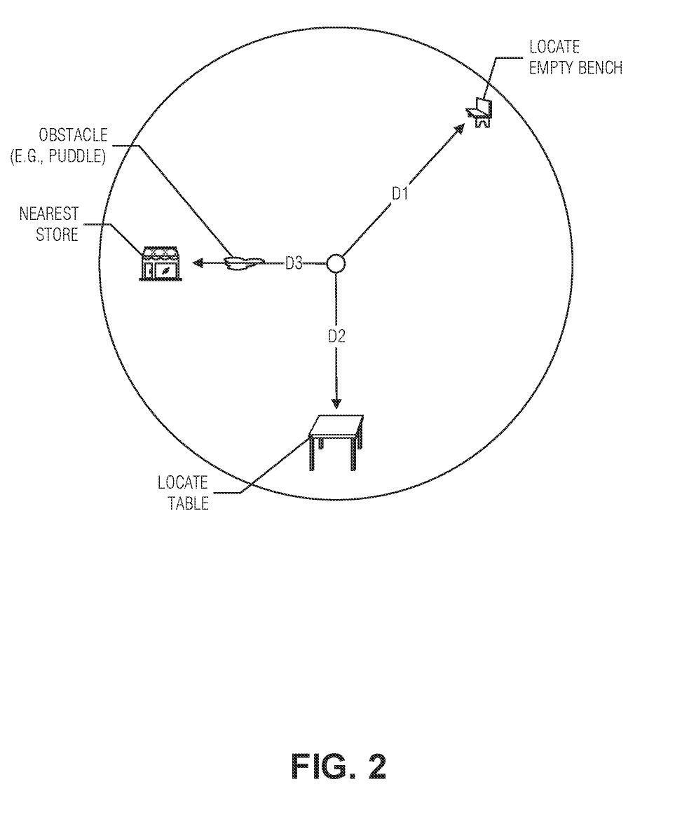

FIG. 2 illustrates an example navigation for a user, according to an embodiment. The user is located at the center of the larger circle. D1, D2, and D3 are paths from the user to an empty bench, a table, and a retail location, respectively. This represents the environment 200 for the user.

Some location based services tend to be focused on things sighted people cannot see, such as local weather information (e.g., forecasts), get travel information (e.g., traffic), get location of nearest stores/restaurants, address of nearest friends, credit card companies use location to prevent fraud, etc. However, visually impaired people may benefit from additional information. In a sense, the AR system translates visual information, as well as other sensor data, to audio and haptic information for consumption by a visually impaired individual.

The example of environment 200 illustrates the information translation concept discussed above. While a sighted person may be able to readily identify the empty bench, the system here converts that visual information into an audio stream informing the user of this fact. Similarly, the other landmarks may be identified visually, or in the case of the business, via a combination of visual data and location data (e.g., GPS). Thus, the AR system provides a “context” to the user by looking around and augmenting the user's reality through audio feedback. For example, a vicinity (e.g., large circle) may be defined as radius of 20-25 meters, similar to a distance pertinent for a sighted person. If am empty bench is not available in the vicinity, the AR system may provide appropriate feedback, such as “No bench available” or note a bench that is occupied but may accommodate one more person, etc.

For example, say the user wants to go to the nearest store selling flowers. There may be obstacles like puddles of water, stones or other objects on the way to the store. The AR system helps the user to successfully avoid these obstacles. Thus, the AR system provides details of the “scene” around the user during navigation towards the destination.

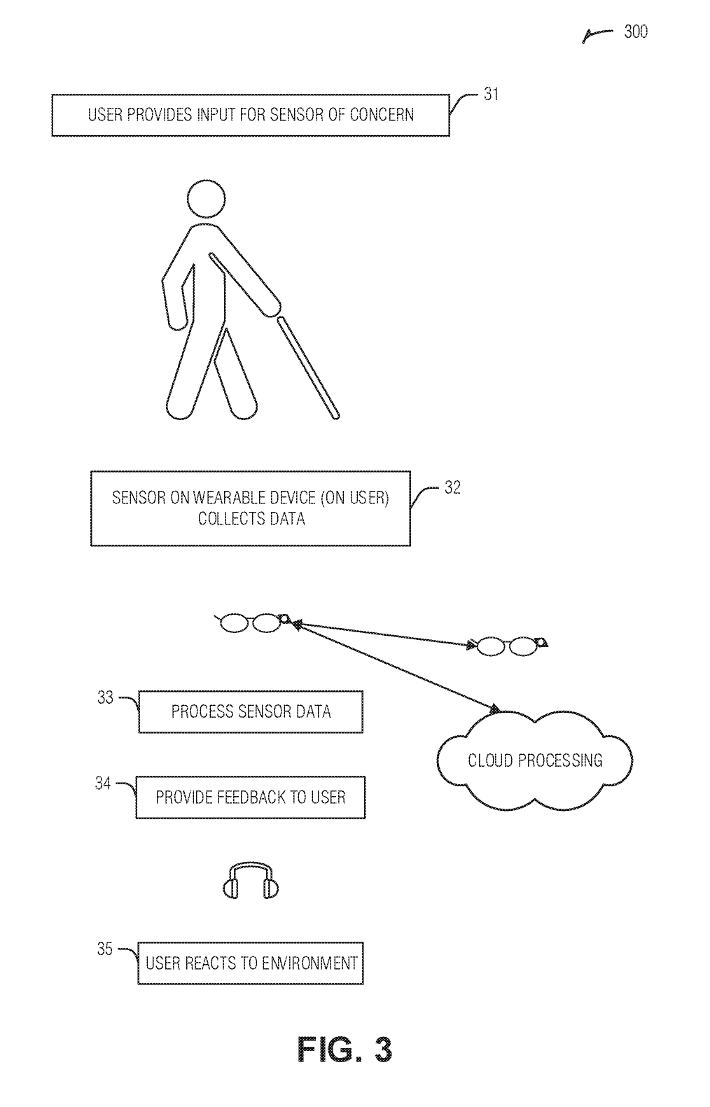

FIG. 3 illustrates an example of a technique 300 to facilitate visually impaired augmented reality, according to an embodiment. A visually impaired user provides voice commands to the device (operation 31). The voice commands are used to determine a task the user wants to accomplish. An example set of commands user can provide to the device may include, but not limited to:
1. Give me directions to “Fancy Pizza”;
2. Tell me about available discounts on the way; and
3. Recognize the faces from my contacts.

At this point, the device is configured and ready to facilitate in the requested task. The device opens a stream to gather data for context awareness (operation 32). Example data as part of the stream (or each may be considered a stream, data feed, etc.) include, but not limited to:
1. A world facing camera on head mount device starts recording frames. This may be used to recognize the environment (e.g. landmarks, parks, pedestrian path etc.). Image data can also be used for face recognition based on the user's contact list of known people, for example.
2. A depth camera to feed data into simultaneous localization and mapping (SLAM) to help navigate the user to destination.
3. A microphone to gathering audio data to classify the environment (e.g. amount of traffic, people around the user, etc.).
4. A pollen sensor or IMU Sensors to augment context awareness.
5. A location sensor to, for example, uses GPS, WLAN, PAN, or other RF signals to assist user in navigating to destination. This may also gather locational awareness (e.g. Fancy Pizza, Coffee Stop deals etc.)

Data is then processed from an array of contextual sensors on the device (operation 33). The data may be processed on the device itself or in the cloud. The choice of local or cloud processing may be made based on connectivity and computational intensity for the task.

The data is processed based on the user's command or the task derived from the user's command. For example, if the user's command was to recognize faces from the contact list, face images collected by the camera are matched with pictures in users contact list; if a match is found, then the user is notified.

The system performs a fusion of data from multiple input streams to generate contextual awareness. For example, data from GPS, WLAN sources, etc. is fed to assist SLAM in navigating the user on the right path for a navigation task (for either indoor or outdoor environments).

In an example, while the device is in active navigation, data is constantly analyzed for personal safety. This may, for instance, detect obstacles in the user's path towards destination. Camera (e.g., visual, infrared, depth, etc.), audio (e.g., from microphones) radar, LIDAR, ultrasonic ranging, satellite navigation, or other sensor data from the device are analyzed for potential dangers, obstacles, people, vehicles, etc. during navigation.

Once the sensor data is processed, user notification (e.g., voice or other audio, haptic) feedback from the device to the user is performed (operation 34). Thus, the information about the environment from operations 32 and 33 are translated to meaningful text and then converted to voice on the device in order to communicate them to the user. For example, given a face recognized from the user's contact list as Patrick. Here, the device creates the text “Patrick is standing 20 feet to your right” based on the sensor data. Another example may relate to user safety, such as “an obstacle detected 10 feet ahead, please take 10 steps towards your left.” After this text is created, a voice engine translates this into speech which the user can hear. Now, after the user hears the feedback from device, the user may react appropriately (event 35).

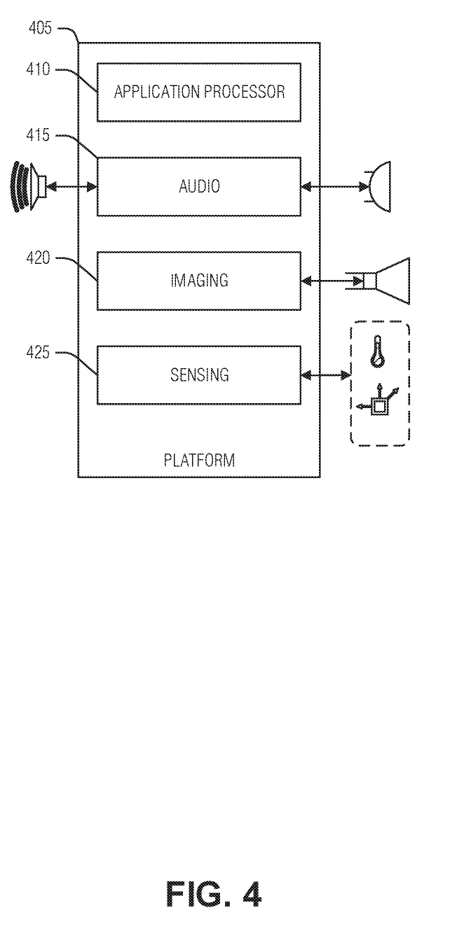

FIG. 4 illustrates an example matrix of a system 405 for visually impaired augmented reality, according to an embodiment. FIG. 4 provides an overall system overview and software stack. The system 405 may include a system on a chip (SoC) 410 to meet performance (e.g., computational and power based) parameters for a wearable device to implement visually impaired AR. The system 405 includes functional blocks to perform audio processing 415, image processing 420, and sensor processing 425 (e.g., handling the sensor inputs from various sensors that are not connected to the audio or image processing engines). The application processor 410 communicates with the functional blocks 415, 420, 425 to implement AR for the visually impaired.

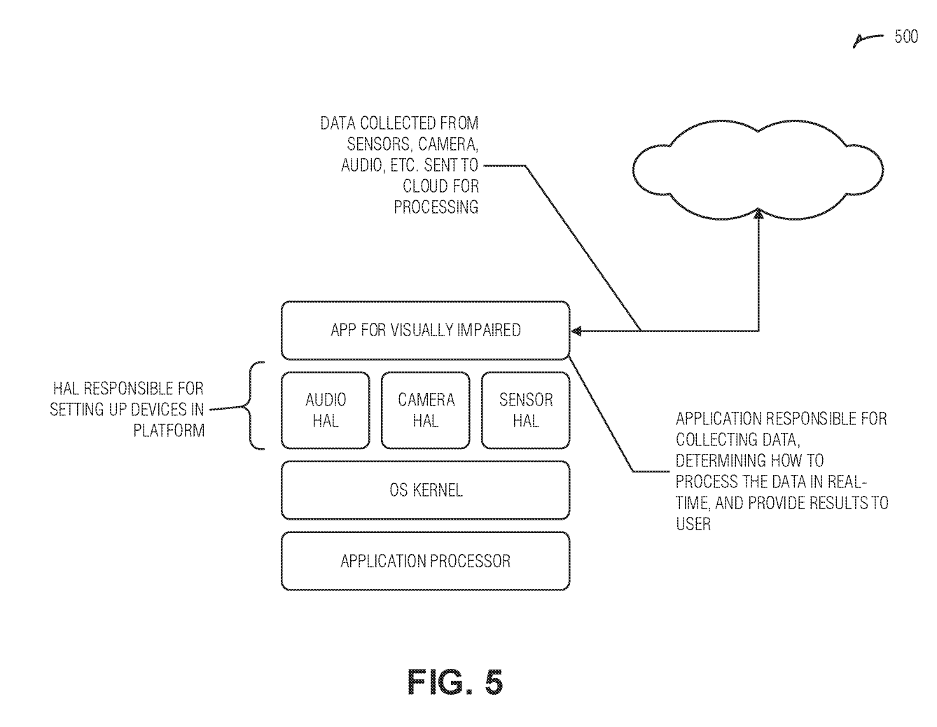

FIG. 5 illustrates an example of a control stack 500 for visually impaired augmented reality, according to an embodiment. The control stack 500, for example, on a device processor, provides the AR experience described herein for visually impaired people. The application for the visually impaired is responsible for coordinating (e.g., orchestrating) the different engines to collect or process sensor inputs. The application is also responsible for communicating with the cloud when the cloud is used. In an example, the application is the only application running on the user's wearable device. That is, in this example, the device is a dedicated device that does not have multiple applications running on the applications processor. Running additional applications may significantly affect the device's performance. Thus, in a dedicated device, only applications used to enable the AR experience are run on the application processor. This has an added benefit of securing the user's privacy as data may be scrubbed or filtered prior to being stored in the cloud where other entities may view the data.

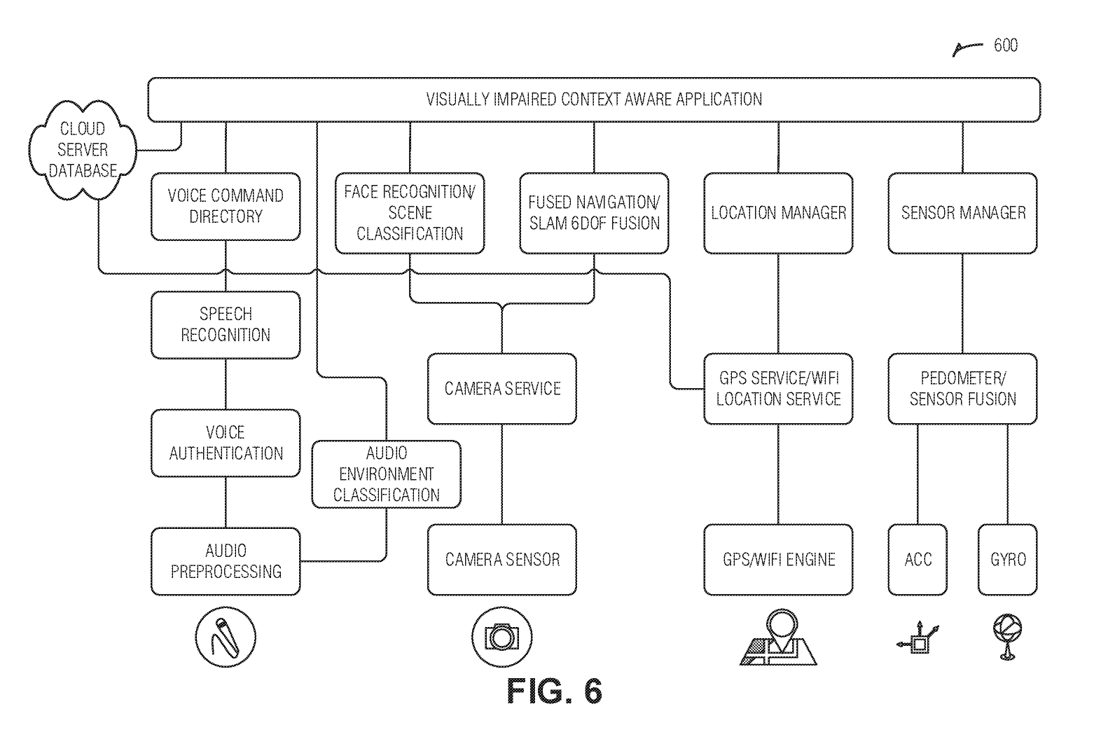

FIG. 6 illustrates an example of a communications flow 600 for visually impaired augmented reality, according to an embodiment. The communication flow 600 is self-explanatory as illustrated. The left-most column is an audio processing pipeline, the next column to the right is a visual processing pipeline, the next column to the right is a location based services pipeline (e.g., navigation), and the right-most column is a motion sensor (e.g., accelerometer and gyrometer) pipeline. The communications flow 600 is an example implementation for a sensor to processor stack, including cloud participation, to implement the AR system described herein.

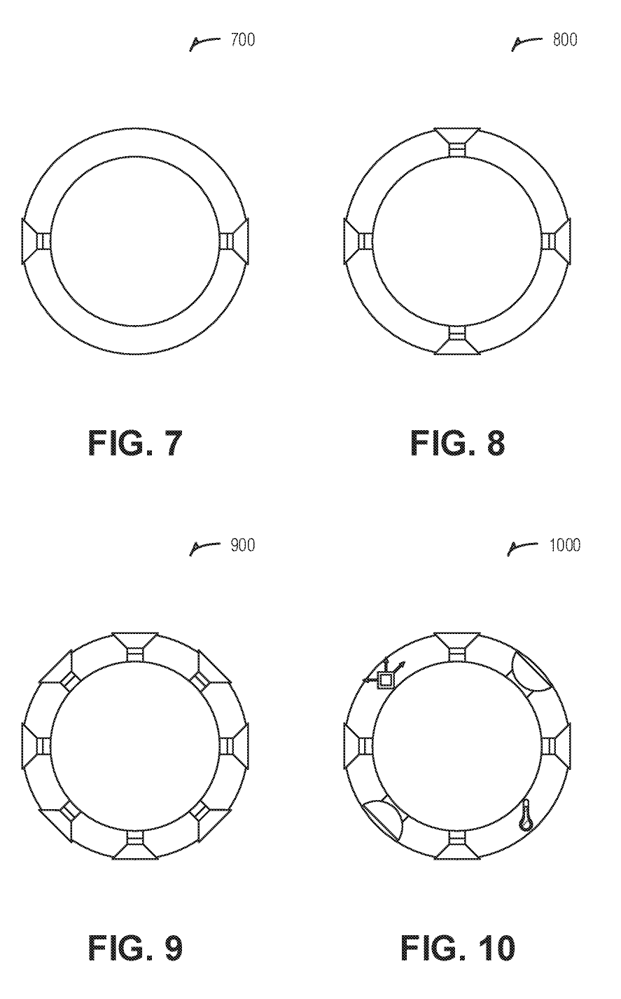

FIGS. 7-10 illustrate several examples of sensor arrangements in a wearable device to facilitate visually impaired augmented reality. FIGS. 7-9 illustrate various camera positions in a ring-shaped wearable device. In an example, the device is arranged to encircle a user's head, like a crown, or may be part of other head worn apparel, such as a hat. In an example, the right facing side is the front of the device and is aligned with the user's face. Thus, device 700 includes two cameras, one each facing forward and backward. Device 800 adds two lateral camera's, one each face left and right of the user. Device 900 adds four more cameras to furnish a diagonal (from the user's perspective) view of the environment. Having multiple cameras allows the AR system to gather visual information from all angles of the environment.

FIG. 10 illustrates a device 1000 that includes sensors other than cameras. Specifically, the device 1000 includes a forward-left diagonally mounted microphone and a rearward-right diagonally mounted microphone along with an accelerometer (rearward-left) and thermometer (forward-right). This configuration allows an efficient distribution of sensing (e.g., audio and visual) to capture the user's environment while also incorporating sensors that are not as sensitive to orientation with respect to the user's facing.

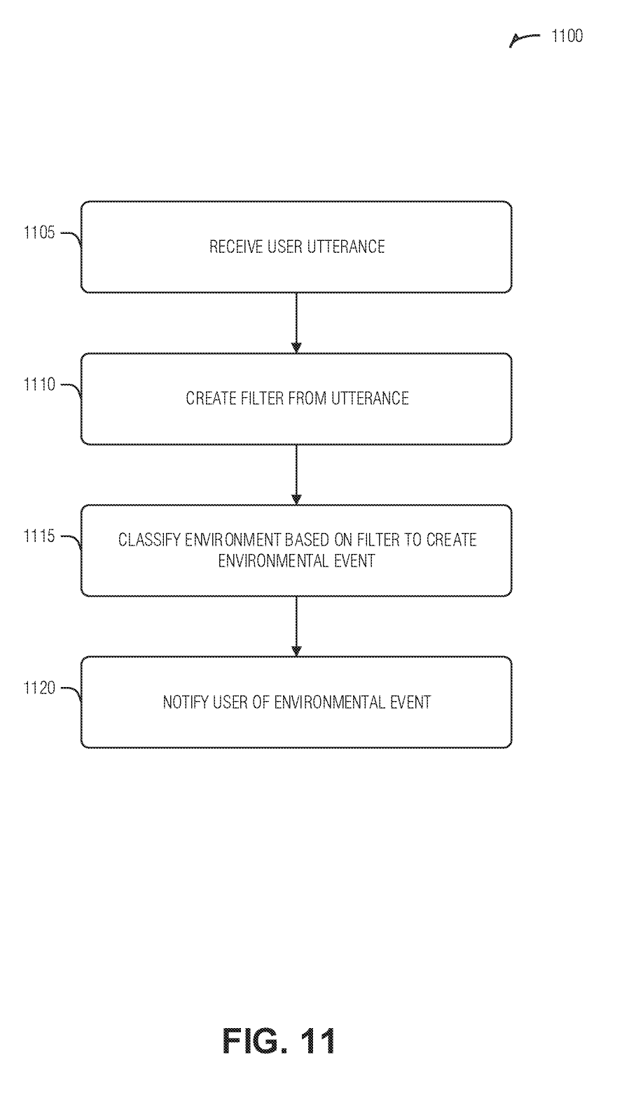

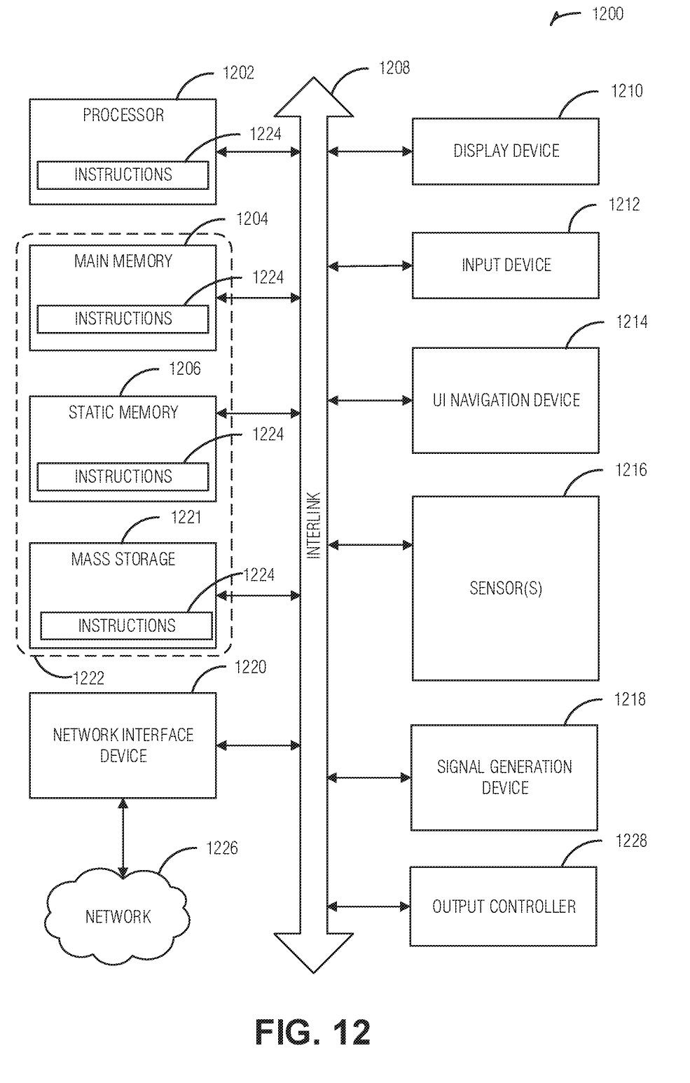

FIG. 11 illustrates a flow diagram of an example of a method 1100 for visually impaired augmented reality, according to an embodiment. The operations of the method 100 are implemented in computer hardware, such as that described above, or below with respect to FIG. 12 (e.g., circuitry).

At operation 1105, an utterance is received from a user.

At operation 1110, the utterance is classified to produce a filter. In an example, the filter is a command to navigate to a destination. In an example, classifying the utterance to produce the filter includes performing a speech-to-text conversion of the utterance to produce a parameter. In an example, classifying the environment of the user based on the filter includes selecting a classifier to classify the environment based on the parameter.

At operation 1115, an environment of the user is classified based on the filter to produce an environmental event. In an example, the environmental event is a waypoint to a destination.

At operation 1120, an audible interpretation of the environmental event is rendered. In an example, the audible interpretation includes a set of words intelligible by the user. In an example, classifying the environment to produce an environmental event includes limiting a frequency of environmental events produced. In an example, the frequency of environmental events produced is based on a cardinality of the set of words corresponding to potential events. In an example, the frequency of environmental events produced is based on a time-to-render of the set of words corresponding to potential events. In an example, the frequency of environmental events produced is beyond a threshold. In an example, the audible interpretation is a generalized description of multiple environmental events.

In an example, the operations of receiving the utterance from the user (operation 1105), classifying the utterance to produce the filter (operation 1110), classifying the environment of the user to produce the environmental event (operation 1115), and rendering the audible interpretation of the environmental event (operation 1120) are performed on the set of devices carried by the user. In an example, the set of devices has a cardinality of one. In an example, the set of devices include at least one of a microphone, a camera, a depth sensor, a distance sensor, a positioning system, a motion sensor, or a context sensor. In an example, the context sensor is at least one of a mapping system, or a short-range radio frequency interrogator.

FIG. 12 illustrates a block diagram of an example machine 1200 upon which any one or more of the techniques (e.g., methodologies) discussed herein may perform. Examples, as described herein, may include, or may operate by, logic or a number of components, or mechanisms in the machine 1200. Circuitry (e.g., processing circuitry) is a collection of circuits implemented in tangible entities of the machine 1200 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1200 follow.

In alternative embodiments, the machine 1200 may operate as a standalone device or may be connected (e.g., networked) to other machines. In a networked deployment, the machine 1200 may operate in the capacity of a server machine, a client machine, or both in server-client network environments. In an example, the machine 1200 may act as a peer machine in peer-to-peer (P2P) (or other distributed) network environment. The machine 1200 may be a wearable device, a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1200 may include a hardware processor 1202 (e.g., a central processing unit (CPU), a graphics processing unit (GPU), a hardware processor core, or any combination thereof), a main memory 1204, a static memory 1206 (e.g., memory or storage for firmware, microcode, a basic-input-output (BIOS), unified extensible firmware interface (UEFI), etc.), and mass storage 1221 (e.g., hard drive, tape drive, flash storage, or other block devices) some or all of which may communicate with each other via an interlink 1208 (e.g., bus). The machine 1200 may further include a display unit 1210, an alphanumeric input device 1212 (e.g., a keyboard), and a user interface (UI) navigation device 1214 (e.g., a mouse). In an example, the display unit 1210, input device 1212 and UI navigation device 1214 may be a touch screen display. The machine 1200 may additionally include a storage device 1216 (e.g., drive unit), a signal generation device 1218 (e.g., a speaker), a network interface device 1220, and one or more sensors 1221, such as a global positioning system (GPS) sensor, compass, accelerometer, or other sensor. The machine 1200 may include an output controller 1228, such as a serial (e.g., universal serial bus (USB)), parallel, or other wired or wireless (e.g., infrared (JR), near field communication (NFC), etc.) connection to communicate or control one or more peripheral devices (e.g., a printer, card reader, etc.).

Registers of the processor 1202, the main memory 1204, the static memory 1206, or the mass storage 1216 may be, or include, a machine readable medium 1222 on which is stored one or more sets of data structures or instructions 1224 (e.g., software) embodying or utilized by any one or more of the techniques or functions described herein. The instructions 1224 may also reside, completely or at least partially, within any of registers of the processor 1202, the main memory 1204, the static memory 1206, or the mass storage 1216 during execution thereof by the machine 1200. In an example, one or any combination of the hardware processor 1202, the main memory 1204, the static memory 1206, or the mass storage 1216 may constitute the machine readable media 1202. While the machine readable medium 1222 is illustrated as a single medium, the term “machine readable medium” may include a single medium or multiple media (e.g., a centralized or distributed database, and/or associated caches and servers) configured to store the one or more instructions 1224.

The term “machine readable medium” may include any medium that is capable of storing, encoding, or carrying instructions for execution by the machine 1200 and that cause the machine 1200 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1224 may be further transmitted or received over a communications network 1226 using a transmission medium via the network interface device 1220 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old. Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1220 may include one or more physical jacks (e.g., Ethernet, coaxial, or phone jacks) or one or more antennas to connect to the communications network 1226. In an example, the network interface device 1220 may include a plurality of antennas to wirelessly communicate using at least one of single-input multiple-output (SIMO); multiple-input multiple-output (MIMO), or multiple-input single-output (MISO) techniques. The term “transmission medium” shall be taken to include an intangible medium that is capable of storing, encoding or carrying instructions for execution by the machine 1200, and includes digital or analog communications signals or other intangible medium to facilitate communication of such software. A transmission medium is a machine readable medium.

### Additional Notes & Examples

Example 1 is a system for visually impaired augmented reality, the system comprising: sensor interface to receive an utterance from a user; a controller to: classify the utterance to produce a filter; and classify an environment of the user based on the filter to produce an environmental event; and an output driver to render an audible interpretation of the environmental event.

In Example 2, the subject matter of Example 1 optionally includes wherein, to classify the utterance to produce the filter, the controller is to perform a speech-to-text conversion of the utterance to produce a parameter.

In Example 3, the subject matter of Example 2 optionally includes wherein, to classify the environment of the user based on the filter, the controller is to select a classifier to classify the environment based on the parameter.

In Example 4, the subject matter of any one or more of Examples 1-3 optionally include wherein the audible interpretation includes a set of words intelligible by the user.

In Example 5, the subject matter of Example 4 optionally includes wherein, to classify the environment to produce an environmental event, the controller is to limit a frequency of environmental events produced.

In Example 6, the subject matter of Example 5 optionally includes wherein the frequency of environmental events produced is based on a cardinality of the set of words corresponding to potential events.

In Example 7, the subject matter of any one or more of Examples 5-6 optionally include wherein the frequency of environmental events produced is based on a time-to-render of the set of words corresponding to potential events.

In Example 8, the subject matter of Example 7 optionally includes wherein the frequency of environmental events produced is beyond a threshold, and wherein the audible interpretation is a generalized description of multiple environmental events.

In Example 9, the subject matter of any one or more of Examples 1-8 optionally include wherein an operation of receiving the utterance from the user, classifying the utterance to produce the filter, classifying the environment of the user to produce the environmental event, and rendering the audible interpretation of the environmental event are performed on the set of devices carried by the user.

In Example 10, the subject matter of Example 9 optionally includes wherein the set of devices has a cardinality of one.

In Example 11, the subject matter of any one or more of Examples 9-10 optionally include wherein the set of devices include at least one of a microphone, a camera, a depth sensor, a distance sensor, a positioning system, a motion sensor, or a context sensor.

In Example 12, the subject matter of Example 11 optionally includes wherein the context sensor is at least one of a mapping system, or a short-range radio frequency interrogator.

In Example 13, the subject matter of any one or more of Examples 1-12 optionally include wherein the filter is a command to navigate to a destination, and wherein the environmental event is a waypoint to the destination.

Example 14 is a machine implemented method for visually impaired augmented reality, the method comprising: receiving an utterance from a user; classifying the utterance to produce a filter; classifying an environment of the user based on the filter to produce an environmental event; and rendering an audible interpretation of the environmental event.

In Example 15, the subject matter of Example 14 optionally includes wherein classifying the utterance to produce the filter includes performing a speech-to-text conversion of the utterance to produce a parameter.

In Example 16, the subject matter of Example 15 optionally includes wherein classifying the environment of the user based on the filter includes selecting a classifier to classify the environment based on the parameter.

In Example 17, the subject matter of any one or more of Examples 14-16 optionally include wherein the audible interpretation includes a set of words intelligible by the user.

In Example 18, the subject matter of Example 17 optionally includes wherein classifying the environment to produce an environmental event includes limiting a frequency of environmental events produced.

In Example 19, the subject matter of Example 18 optionally includes wherein the frequency of environmental events produced is based on a cardinality of the set of words corresponding to potential events.

In Example 20, the subject matter of any one or more of Examples 18-19 optionally include wherein the frequency of environmental events produced is based on a time-to-render of the set of words corresponding to potential events.

In Example 21, the subject matter of Example 20 optionally includes wherein the frequency of environmental events produced is beyond a threshold, and wherein the audible interpretation is a generalized description of multiple environmental events.

In Example 22, the subject matter of any one or more of Examples 14-21 optionally include wherein an operation of receiving the utterance from the user, classifying the utterance to produce the filter, classifying the environment of the user to produce the environmental event, and rendering the audible interpretation of the environmental event are performed on the set of devices carried by the user.

In Example 23, the subject matter of Example 22 optionally includes wherein the set of devices has a cardinality of one.

In Example 24, the subject matter of any one or more of Examples 22-23 optionally include wherein the set of devices include at least one of a microphone, a camera, a depth sensor, a distance sensor, a positioning system, a motion sensor, or a context sensor.

In Example 25, the subject matter of Example 24 optionally includes wherein the context sensor is at least one of a mapping system, or a short-range radio frequency interrogator.

In Example 26, the subject matter of any one or more of Examples 14-25 optionally include wherein the filter is a command to navigate to a destination, and wherein the environmental event is a waypoint to the destination.

Example 27 is at least one machine readable medium including instructions that, when performed by processing circuitry, cause the processing circuitry to perform any method of Examples 14-26.

Example 28 is a system including means to perform any method of Examples 14-26.

Example 29 is at least one machine readable medium including instructions for visually impaired augmented reality, the instructions, when executed by processing circuitry, cause the processing circuitry to perform operations comprising: receiving an utterance from a user; classifying the utterance to produce a filter; classifying an environment of the user based on the filter to produce an environmental event; and rendering an audible interpretation of the environmental event.

In Example 30, the subject matter of Example 29 optionally includes wherein classifying the utterance to produce the filter includes performing a speech-to-text conversion of the utterance to produce a parameter.

In Example 31, the subject matter of Example 30 optionally includes wherein classifying the environment of the user based on the filter includes selecting a classifier to classify the environment based on the parameter.

In Example 32, the subject matter of any one or more of Examples 29-31 optionally include wherein the audible interpretation includes a set of words intelligible by the user.

In Example 33, the subject matter of Example 32 optionally includes wherein classifying the environment to produce an environmental event includes limiting a frequency of environmental events produced.

In Example 34, the subject matter of Example 33 optionally includes wherein the frequency of environmental events produced is based on a cardinality of the set of words corresponding to potential events.

In Example 35, the subject matter of any one or more of Examples 33-34 optionally include wherein the frequency of environmental events produced is based on a time-to-render of the set of words corresponding to potential events.

In Example 36, the subject matter of Example 35 optionally includes wherein the frequency of environmental events produced is beyond a threshold, and wherein the audible interpretation is a generalized description of multiple environmental events.

In Example 37, the subject matter of any one or more of Examples 29-36 optionally include wherein an operation of receiving the utterance from the user, classifying the utterance to produce the filter, classifying the environment of the user to produce the environmental event, and rendering the audible interpretation of the environmental event are performed on the set of devices carried by the user.

In Example 38, the subject matter of Example 37 optionally includes wherein the set of devices has a cardinality of one.

In Example 39, the subject matter of any one or more of Examples 37-38 optionally include wherein the set of devices include at least one of a microphone, a camera, a depth sensor, a distance sensor, a positioning system, a motion sensor, or a context sensor.

In Example 40, the subject matter of Example 39 optionally includes wherein the context sensor is at least one of a mapping system, or a short-range radio frequency interrogator.

In Example 41, the subject matter of any one or more of Examples 29-40 optionally include wherein the filter is a command to navigate to a destination, and wherein the environmental event is a waypoint to the destination.

Example 42 is a system for visually impaired augmented reality, the system comprising: means for receiving an utterance from a user; means for classifying the utterance to produce a filter; means for classifying an environment of the user based on the filter to produce an environmental event; and means for rendering an audible interpretation of the environmental event.

In Example 43, the subject matter of Example 42 optionally includes wherein the means for classifying the utterance to produce the filter includes means for performing a speech-to-text conversion of the utterance to produce a parameter.

In Example 44, the subject matter of Example 43 optionally includes wherein the means for classifying the environment of the user based on the filter includes means for selecting a classifier to classify the environment based on the parameter.

In Example 45, the subject matter of any one or more of Examples 42-44 optionally include wherein the audible interpretation includes a set of words intelligible by the user.

In Example 46, the subject matter of Example 45 optionally includes wherein the means for classifying the environment to produce an environmental event includes means for limiting a frequency of environmental events produced.

In Example 47, the subject matter of Example 46 optionally includes wherein the frequency of environmental events produced is based on a cardinality of the set of words corresponding to potential events.

In Example 48, the subject matter of any one or more of Examples 46-47 optionally include wherein the frequency of environmental events produced is based on a time-to-render of the set of words corresponding to potential events.

In Example 49, the subject matter of Example 48 optionally includes wherein the frequency of environmental events produced is beyond a threshold, and wherein the audible interpretation is a generalized description of multiple environmental events.

In Example 50, the subject matter of any one or more of Examples 42-49 optionally include wherein an operation of receiving the utterance from the user, classifying the utterance to produce the filter, classifying the environment of the user to produce the environmental event, and rendering the audible interpretation of the environmental event are performed on the set of devices carried by the user.

In Example 51, the subject matter of Example 50 optionally includes wherein the set of devices has a cardinality of one.

In Example 52, the subject matter of any one or more of Examples 50-51 optionally include wherein the set of devices include at least one of a microphone, a camera, a depth sensor, a distance sensor, a positioning system, a motion sensor, or a context sensor.

In Example 53, the subject matter of Example 52 optionally includes wherein the context sensor is at least one of a mapping system, or a short-range radio frequency interrogator.

In Example 54, the subject matter of any one or more of Examples 42-53 optionally include wherein the filter is a command to navigate to a destination, and wherein the environmental event is a waypoint to the destination.

Example 55 is at least one machine-readable medium including instructions, which when executed by a machine, cause the machine to perform operations of any of the operations of Examples 1-54.

Example 56 is an apparatus comprising means for performing any of the operations of Examples 1-55.

Example 57 is a system to perform the operations of any of the Examples 1-54.

Example 58 is a method to perform the operations of any of the Examples 1-54.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1. A system for visually impaired augmented reality, the system comprising:
sensor interface to receive an utterance from a user;
a controller to:
classify the utterance to produce a filter; and
classify an environment of the user based on the filter to produce an environmental event, wherein, to classify the environment, the controller selects a classifier from multiple classifiers based on the filter; and
an output driver to render an audible interpretation of the environmental event, wherein the system is implemented in circuitry of a user worn device or a user held device.

2. The system of claim 1, wherein, to classify the utterance to produce the filter, the controller is to perform a speech-to-text conversion of the utterance to produce a parameter.

3. The system of claim 2, wherein, to classify the environment of the user based on the filter, the controller is to select a classifier to classify the environment based on the parameter.

4. The system of claim 1, wherein the audible interpretation includes a set of words intelligible by the user.

5. The system of claim 4, wherein, to classify the environment to produce an environmental event, the controller is to limit a frequency of environmental events produced.

6. The system of claim 5, wherein the frequency of environmental events produced is based on a cardinality of the set of words corresponding to potential events.

7. The system of claim 5, wherein the frequency of environmental events produced is based on a time-to-render of the set of words corresponding to potential events.

8. The system of claim 7, wherein the frequency of environmental events produced is beyond a threshold, and wherein the audible interpretation is a generalized description of multiple environmental events.

9. A machine implemented method for visually impaired augmented reality, the method comprising:
receiving, by a sensor interface of the machine, an utterance from a user;
classifying, by a controller of the machine, the utterance to produce a filter;
classifying, by the controller, an environment of the user based on the filter to produce an environmental event, wherein classifying the environment includes selecting a classifier from multiple classifiers based on the filter; and
rendering, by an output driver of the machine, an audible interpretation of the environmental event, wherein the machine is implemented in circuitry of a user worn device or a user held device.

10. The method of claim 9, wherein classifying the utterance to produce the filter includes performing a speech-to-text conversion of the utterance to produce a parameter.

11. The method of claim 10, wherein classifying the environment of the user based on the filter includes selecting a classifier to classify the environment based on the parameter.

12. The method of claim 9, wherein the audible interpretation includes a set of words intelligible by the user.

13. The method of claim 12, wherein classifying the environment to produce an environmental event includes limiting a frequency of environmental events produced.

14. The method of claim 13, wherein the frequency of environmental events produced is based on a cardinality of the set of words corresponding to potential events.

15. The method of claim 13, wherein the frequency of environmental events produced is based on a time-to-render of the set of words corresponding to potential events.

16. The method of claim 15, wherein the frequency of environmental events produced is beyond a threshold, and wherein the audible interpretation is a generalized description of multiple environmental events.

17. At least one non-transitory machine readable medium including instructions for visually impaired augmented reality, the instructions, when executed by processing circuitry, cause the processing circuitry to perform operations comprising:
receiving an utterance from a user;
classifying the utterance to produce a filter;
classifying an environment of the user based on the filter to produce an environmental event, wherein classifying the environment includes selecting a classifier from multiple classifiers based on the filter; and
rendering an audible interpretation of the environmental event, wherein the processing circuitry is in a user worn device or a user held device.

18. The at least one machine readable medium of claim 17, wherein classifying the utterance to produce the filter includes performing a speech-to-text conversion of the utterance to produce a parameter.

19. The at least one machine readable medium of claim 18, wherein classifying the environment of the user based on the filter includes selecting a classifier to classify the environment based on the parameter.

20. The at least one machine readable medium of claim 17, wherein the audible interpretation includes a set of words intelligible by the user.

21. The at least one machine readable medium of claim 20, wherein classifying the environment to produce an environmental event includes limiting a frequency of environmental events produced.

22. The at least one machine readable medium of claim 21, wherein the frequency of environmental events produced is based on a cardinality of the set of words corresponding to potential events.

23. The at least one machine readable medium of claim 21, wherein the frequency of environmental events produced is based on a time-to-render of the set of words corresponding to potential events.

24. The at least one machine readable medium of claim 23, wherein the frequency of environmental events produced is beyond a threshold, and wherein the audible interpretation is a generalized description of multiple environmental events.
